(19) United States
(12) Patent Application Publication
(10) Pub. No.: US 2022/0128560 A1
(43) Pub. Date: Apr. 28, 2022

(54) HIGH THROUGHOUT IMMUNOASSAYS AND METHODS FOR THE DETECTION OF SARS-COV-2 ANTIGENS

(71) Applicant: Ortho-Clinical Diagnostics, Inc., Raritan, NJ (US)

(72) Inventors: Philip Hosimer, Raritan, NJ (US); Charles A. Noeson, Raritan, NJ (US); Michael T. Colvin, Raritan, NJ (US); Amelia Brunner, Raritan, NJ (US); Sierra Clark, Raritan, NJ (US); Paul Contestable, Raritan, NJ (US)

(21) Appl. No.: 17/513,538

(22) Filed: Oct. 28, 2021

Related U.S. Application Data
(60) Provisional application No. 63/106,830, filed on Oct. 28, 2020.

(51) Int. Cl. G01N 33/569

(52) U.S. Cl. CPC G01N 33/56983; G01N 2333/165

(57) ABSTRACT

Disclosed herein are methods, reagents and devices for the rapid detection of SARS-CoV-2 nucleocapsid antigens in clinical samples using high throughput methods.

### CROSS-REFERENCE TO RELATED APPLICATIONS

The present application claims the benefit of U.S. Provisional Patent Application 63/106,830 filed Oct. 28, 2021, the entire contents of which are incorporated by reference herein in its entirety.

### FIELD

The present disclosure generally relates to immunoassays and related methods for the detection of viral antigens. More specifically the present disclosure relates to SARS-CoV-2 viral antigen detection using automated and semi-automated high through-put immunoassays.

### BACKGROUND

On Mar. 11, 2020, the World Health Organization declared the coronavirus disease (SARS-CoV-2 aka COVID-19) outbreak a pandemic. Since the disease was first reported in late December 2019 in Wuhan, China, it has spread to more than 216 countries and territories globally. Rapid viral detection is a tool of paramount importance in containing the virus spread. Antigen detection allows epidemiologist and public health authorities to establish with a high level of confidence those individuals capable of spreading the disease even when asymptomatic. However, testing the tens of thousands of suspected carriers, symptomatic persons and asymptomatic persons in susceptible populations cannot be accomplished using manual methods and rapid tests such as lateral flow devices and the like. Therefore there is an urgent need for high specificity, high sensitivity immunoassays using automated and semi-automated immunoassays devices such as the Ortho Clinical Diagnostics VITROS® (registered trademark of Crimson U.S. Assets LLC Limited Liability Company Delaware 1001 US Route 202 Raritan New Jersey 08869) Immunodiagnostic Products.

### SUMMARY

This disclosure provides immunoassays useful for detecting SARS-CoV-2 antigens in clinical samples.

Thus, disclosed herein are methods for the high thoughput testing of samples for the presence of SARS-CoV-2 nucleocapsid comprising: providing a immunoassay system capable of performing high throughput assays; reacting a patient sample with a capture antibody that recognizes the SARS-CoV-2 nucleocapsid, a detection antibody that recognizes the SARS-CoV-2 nucleocapsid at a location different from the capture antibody, and a means for detecting a complex comprising the capture antibody, the detection antibody, and the SARS-CoV-2 nucleocapsid; and detecting the complex comprising the capture antibody, the detection antibody, and the SARS-CoV-2 nucleocapsid; wherein the reacting and detecting steps are performed in the immunoassay system.

Also disclosed herein are kits for the high thoughput testing of patient samples for the presence of SARS-CoV-2 nucleocapsid in a high throughput immunoassay system comprising: a capture antibody that recognizes the SARS-CoV-2 nucleocapsid and a solid support associated therewith; a detection antibody that recognizes the SARS-CoV-2 nucleocapsid at a location difference from the capture antibody; a means for detecting a complex comprising the capture antibody, the detection antibody, and the SARS-CoV-2 nucleocapsid; and instructions for performing the assay.

In some embodiments the immunoassay system capable of performing high throughput assays is an Ortho Clinical Diagnostics VITROS® device.

In some embodiments, the capture antibody is associated with a solid support. In some embodiments, the capture antibody is a monoclonal antibody specific for the SARS-CoV-2 nucleocapsid. In some embodiments, the capture antibody is a rabbit monoclonal antibody. In some embodiments, the detection antibody is a monoclonal antibody specific for the SARS-CoV-2 nucleocapsid. In some embodiments, the detection antibody is a mouse monoclonal antibody.

In some embodiments, the capture antibody and detection antibody do not cross-react with coronaviruses other than SARS-CoV-1. In some embodiments, the detection antibody is in a conjugate, wherein each conjugate comprises about 100 horseradish peroxidase (HRP) molecules and 25 immunoglobulin molecules.

In some embodiments, the patient sample is a nasopharyngeal swab or an anterior nasal swab.

Additional features and advantages of the subject disclosure will be apparent from the description which follows when considered in conjunction with the attached figures.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every FIGURE, nor is every component of each embodiment of the invention shown where illustration is not necessary to allow those of ordinary skill in the art to understand the invention.

FIG. 1 depicts the general SARS-CoV-2 antigen assay structure of the present disclosure.

### DETAILED DESCRIPTION

Disclosed herein is a high-throughput assay to detect the presence of SARS-CoV-2 virus in patient samples, including nasopharyngeal swab, nasal swab samples, such an anterior nasal swab samples, and saliva samples from patients infected with, or suspected of infection with, SARS-CoV-2, the virus associated with the disease known as COVID-19.

High-throughput means a method used in the fields of biology and chemistry using robotics, data processing/control software, liquid handling devices, and sensitive detectors, high-throughput screening allows a clinical laboratory scientist to quickly conduct hundred, thousands or even millions of chemical, genetic, or pharmacological tests in a short time period such as an hour or a day. Through this process one can rapidly identify active compounds, antibodies, or genes involved in a disease process.

In embodiments of the present disclosure, the assay is in performed in a high throughout assay device such as the VITROS® system (Ortho-Clinical Diagnostics, Raritan, N.J.). See U.S. Pat. No. 7,250,303, the entire contents of which are incorporated herein by reference for all it teaches related immunoassays devices and methods. Non-limiting examples of high throughput devices include VITROS® XT 7600 and 5600 Integrated Systems, the VITROS® 3600 Immunodiagnostic System, and equivalents thereof.

One method of testing samples for SARS-CoV-2 involves using real-time polymerase chain reaction (RT-PCR) to detect the presence of SARS-CoV-2 ribonucleic acid (RNA). Another method is to detect protein antigens associated with the virus. Several such tests have been introduced by diagnostic companies experienced in developing tests for other respiratory viruses, such as influenza, RSV, or SARS-CoV-1. Antigen tests generally detect nucleocapsid antigen, as it is unique, relatively stable to mutation, and present at a large copy number. The copy number (number of proteins present in each virus particle) is believed to be about 1000 nucleoprotein molecules per virus. Nucleoprotein is also known as nucleocapsid protein.

Upper respiratory specimens, such as nasopharyngeal swabs or anterior nasal swab are useful for diagnostic testing. In some embodiments, saliva samples are also useful for detecting SARS-CoV-2 antigens.

The present inventors have developed a test which includes a lysis buffer manual pre-treatment step, followed by testing on the VITROS® Immunodiagnostics analyzer which uses chemiluminescent detection. The VITROS® assay protocol utilizes a two-step immunometric assay to detect the antigen after the virus has been lysed with the pre-treatment protocol. The immunometric assay uses an antibody pair comprising a capture antibody and a detection antibody, both antibodies specific for the SARS-CoV-2 nucleoprotein. In certain embodiments, the two antibodies can be monoclonal or polyclonal antibodies and can be from a species selected from mouse, rat, goat, rabbit, or any other species from which monoclonal antibodies can be produced. In some embodiments, the capture antibody is a rabbit anti-SARS-CoV-2 nucleoprotein monoclonal antibody and the detection antibody is a mouse anti-SARS-CoV-2 nucleoprotein monoclonal antibody, however the species from which the respective antibodies are derived is non-limiting. In some embodiments, the antibodies are produced from a recombinant expression system. In some embodiments, the antibodies can cross-react with the SARS-CoV-1 coronavirus, but do not cross react with other coronaviruses.

A feature of the assay reagents is the use of a poly-horseradish peroxidase (HRP) detection antibody conjugate to amplify the detection signal. Each conjugate molecule is estimated to contain about 100 HRP molecules and 25 immunoglobulin molecules, so each binding event generates up to 20 times the signal of a standard HRP conjugate. Typical HRP conjugates contain one immunoglobulin molecule covalently attached to 2-5 HRP molecules.

The presently disclosed assay is an immunometric technique involving a two stage reaction. In the first stage, SARS-CoV-2 nucleocapsid antigen present in the sample binds with monoclonal anti-SARS-CoV-2 coated on a microwell. Unbound sample is removed by washing. In the second stage HRP-labeled monoclonal anti-SARS-CoV-2 is added in the conjugate reagent. The conjugate binds specifically to any SARS-CoV-2 nucleocapsid captured on the well in the first stage. Unbound conjugate is removed by the subsequent wash step. The bound HRP conjugate is measured by a luminescent reaction. A reagent containing luminogenic substrates (a luminol derivative and a peracid salt) and an electron transfer agent is added to the wells. The HRP in the bound conjugate catalyzes the oxidation of the luminol derivative, producing light. The electron transfer agent (a substituted acetanilide) increases the level of light produced and prolongs its emission. Signal to cutoff numerical values will increase as the amount of SARS-CoV-2 antigen present in the sample increases.

### Example 1. Assay Procedure

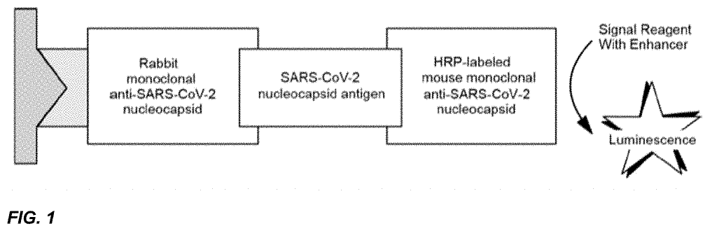

The assay procedure is described below and in FIG. 1.
1. Collect nasal or nasopharyngeal swab sample from the patient, and deposit in a tube of 1-3 mL Viral Transport Medium (VTM) for short-term storage up to three days.
2. Pretreat the sample to lyse the virus by combining 400 μL of the VTM fluid surrounding the swab sample with 100 μL of 10× Lysis Buffer. The VTM/Lysis Buffer mixture is vortexed and then placed on the VITROS® analyzer for immediate testing.
3. On the analyzer, the following protocol is executed.
   a. 80 μL sample (4 parts VTM+1 part 10× Lysis Buffer) and 20 μL Assay Reagent (diluent containing pH buffer and protein) are combined in a coated microwell
   b. Incubate at 37° C. for 30 min.
   c. Aspirate the well and wash.
   d. Add 125 μL HRP Conjugate Reagent (containing the poly-HRP conjugated to detection antibody in a pH buffer with protein).
   e. Incubate 8 min 37° C.
   f. Add 100 μL Signal Reagent (50 μL SR-A plus 50 μL SR-B)
   g. Incubate 5 min, then read signal in luminometer.

The coated microwells comprise streptavidin-coated wells that have been overcoated with a biotin-labeled capture antibody directed against SARS CoV-2 nucleoprotein. The overcoat step applies a coating solution of 140 μL/well with a biotin conjugate concentration of 1 mg/kg. After an overnight incubation the wells are washed, then overcoated with a protein-sugar solution (TSSB), then dried and packed into reagent packs.

The assay is calibrated using recombinant SARS-CoV-2 nucleocapsid antigen formulated in Phosphate Buffered Saline (PBS) with 3% bovine serum albumin (BSA) stored frozen at −20° C.

### Example 2. Determination of Limit of Detection

The Limit of Detection (LoD) was determined by evaluating different dilutions of heat inactivated SARS-CoV-2 virus added to pooled nasal wash. 50 μL of the viral particle solution was added to dry swabs and the swab was then placed into 2 mL of transport media. The transport media with eluted viral particles was tested repeatedly using the VITROS® SARS-CoV-2 Antigen test (n=20). LoD is defined as the lowest virus concentration at which a minimum of 19 replicates out of 20 generate a Reactive result. Testing was performed across seven transport media types and the resulting LoD ranged from 5.0×10² TCID₅₀ (median tissue culture infectious dose) per mL to 3.0×10³ TCID₅₀ per mL (Table 1).

TABLE 1
LoD Determinations

| Transport Medium | TCID₅₀ per mL |
|---|---|
| CDC Viral Transport Medium | 5.0 × 10² |
| COPAN Universal Transport Medium | 5.0 × 10² |
| Hardy Viral Transport Medium | 1.5 × 10³ |
| FlexTrans Transport Medium | 3.0 × 10³ |
| WHO Viral Transport Medium | 7.6 × 10² |
| Saline (PBS and 0.9% NaCl) | 1.5 × 10³ |

### Example 3. Clinical Performance Characteristics with Nasopharyngeal Specimens

Clinical performance characteristics of the VITROS® SARS-CoV-2 Antigen test were evaluated using residual samples from patients suspected of having contracted the SARS-CoV-2 virus within seven days of symptom onset. Samples were collected during two clinical trials as follows:

TABLE 2
Patient characteristics

| | Study 1 | Study 2 |
|---|---|---|
| Female | 69 | 41 |
| Male | 39 | 111 |
| Less than 21 yrs old | 7 | 6 |
| 21-60 yrs old | 47 | 116 |
| Over 60 yrs old | 51 | 30 |

Nasopharyngeal samples were stored frozen between the time of collection and the time of testing. FDA Emergency Use Authorized high sensitivity real-time Polymerase Chain Reaction (RT-PCR) assays for the detection of SARS-CoV-2 were utilized as the comparator methods for these studies.

Testing was performed by operators who were blinded to the RT-PCR test result. External control testing, using VITROS® SARS-CoV-2 Antigen Controls was performed on each day of VITROS® testing. The performance of VITROS® SARS-CoV-2 Antigen test was established with 105 nasopharyngeal specimens collected from individual symptomatic patients (within 7 days of onset) who were suspected of COVID-19 and compared to RT-PCR on a paired NP swab.

TABLE 2A
VITROS ® SARS-CoV-2 Antigen Performance in RT-PCR Positive
and Negative Nasopharyngeal Samples Collected Within 7 Days
of Symptom Onset Against the Comparator Method-Study 1

| VITROS ® SARS- | RT-PCR Comparator Method | | |
|---|---|---|---|
| CoV-2 Antigen Test | Detected | Not Detected | Total |
| Reactive | 24 | 0 | 24 |
| Non-reactive | 6 | 75 | 81 |
| Total | 30 | 75 | 105 |

Positive Percent Agreement (PPA): 80.0% (95% Cl: 56.6-88.5%)
Negative Percent Agreement (NPA): 100.0% (95% Cl: 95.2-100.0%)

TABLE 2B
Positive results broken down by days
since symptom onset-Study 1

| Days Since | Cumulative | Cumulative | |
|---|---|---|---|
| Symptom | VITROS ® PCR | VITROS ® | |
| Onset | Positive (+) | Reactive (+) | PPA |
| 0 | 8 | 4 | 50.0% |
| 1 | 10 | 6 | 60.0% |
| 2 | 14 | 10 | 71.4% |
| 3 | 20 | 16 | 80.0% |
| 4 | 20 | 16 | 80.0% |
| 5 | 22 | 18 | 81.8% |
| 6 | 26 | 21 | 80.8% |
| 7 | 30 | 24 | 80.0% |

TABLE 3A
VITROS ® SARS-CoV-2 Antigen Performance in RT-PCR Positive
and Negative Nasopharyngeal Samples Collected Within 7 Days
of Symptom Onset Against the Comparator Method-Study 2

| VITROS ® SARS- | RT-PCR Comparator Method | | |
|---|---|---|---|
| CoV-2 Antigen Test | Detected | Not Detected | Total |
| Reactive | 56 | 2** | 58 |
| Non-reactive | 9* | 85 | 94 |
| Total | 65 | 87 | 152 |

PPA: 86.2% (95% Cl: 75.3-93.5%)
NPA: 100.0% (95% Cl: 91.9-99.7%)
*One non-reactive result was also negative on an alternate RT-PCR method
**One reactive result was also positive on an alternate RT-PCR method

TABLE 3B
Positive results broken down by days since symptom onset-Study 2

| Days Since | Cumulative | Cumulative | |
|---|---|---|---|
| Symptom | VITROS ® | VITROS ® | |
| Onset | PCR Positive (+) | Reactive (+) | PPA |
| 0 | 2 | 2 | 100.0% |
| 1 | 8 | 8 | 100.0% |
| 2 | 19 | 19 | 100.0% |
| 3 | 34 | 31 | 91.2% |
| 4 | 40 | 36 | 90.0% |
| 5 | 55 | 47 | 85.5% |
| 6 | 63 | 54 | 85.7% |
| 7 | 65 | 56 | 86.2% |

The performance of the VITROS® SARS-CoV-2 Antigen test with positive results stratified by the comparator method cycle threshold (Ct) counts were collected and assessed to better understand the correlation of assay performance to the cycle threshold.

TABLE 4

| VITROS ® | Comparator Method | |
|---|---|---|
| SARS-CoV-2 | (Positive by Ct category) | |
| Antigen Test | Positive (<30 Ct) | Positive (≥30 Ct) |
| Reactive | 55 | 1 |
| Non-reactive | 3 | 6* |
| PPA | 94.8% | 14.3% |
| Positive | 87.5-99.6% | 0.4-57.9% |
| Agreement (95% Cl) | | |

Ct values are not standardized between RT-PCR assays and cannot be compared between assays. Ct values should are not used to determine a patient's viral load, how infectious a person may be, or when a person can be released from isolation or quarantine. Therefore, a negative result from the VITROS® SARS-CoV-2 Antigen test does not establish that an individual is not infectious, and should not be used to make isolation or infection control decisions. All negative results are presumptive for the diagnosis of SARS-CoV-2 and may need to be confirmed with a molecular SARS-CoV-2 assay.

### Example 4. Clinical Performance Characteristics with Anterior Nasal Specimens

Clinical performance characteristics of the VITROS® SARS-CoV-2 Antigen test was evaluated using residual samples from patients suspected of having contracted the SARS-CoV-2 virus within seven days of symptom onset. Samples were collected from 41 female patients and 111 male patients. Six samples were from patients less than 21 years of age, 116 were from patients 21 to 60 years of age and 30 were from patients over the age of 60. Nasal samples were stored frozen between the time of collection and the time of testing. FDA Emergency Use Authorized high sensitivity RT-PCR assays for the detection of SARS-CoV-2 were utilized as the comparator methods for this study.

Testing was performed by operators who were blinded to the RT-PCR test result. External control testing, using VITROS® SARS-CoV-2 Antigen Controls was performed on each day of VITROS® testing. The performance below of VITROS® SARS-CoV-2 Antigen test was established with 152 nasal specimens collected from individual symptomatic patients (within 7 days of onset) who were suspected of COVID-19 and compared to RT-PCR on a paired anterior nasal swab. Performance compared to a paired RT-PCR on an NP swab is also presented in the Table 5A-B below.

TABLE 5A
VITROS ® SARS-CoV-2 Antigen Performance in RT-PCR
Positive and Negative Nasal Samples Collected Within
7 Days of Symptom Onset Against the Comparator Method

| VITROS ® SARS- | RT-PCR Comparator Method | | |
|---|---|---|---|
| CoV-2 Antigen Test | Detected | Not Detected | Total |
| Reactive | 49 | 0 | 49 |
| Non-reactive | 10* | 93 | 103 |
| Total | 59 | 93 | 152 |

PPA: 83.1% (95% Cl: 71.0-91.6%)
NPA: 100.0% (95% Cl: 96.1-100.0%)
*Two non-reactive results were also negative on an alternate RT-PCR method

TABLE 5B
Positive results broken down by days since symptom onset

| Days Since | Cumulative | Cumulative | |
|---|---|---|---|
| Symptom | VITROS ® | VITROS ® | |
| Onset | PCR Positive (+) | Reactive (+) | PPA |
| 0 | 2 | 1 | 50.0% |
| 1 | 8 | 7 | 87.5% |
| 2 | 18 | 17 | 94.4% |
| 3 | 31 | 29 | 93.5% |
| 4 | 35 | 32 | 91.4% |
| 5 | 49 | 42 | 85.7% |
| 6 | 57 | 47 | 82.5% |
| 7 | 59 | 49 | 83.1% |

The performance of the VITROS® SARS-CoV-2 Antigen test with positive results stratified by the comparator method cycle threshold (Ct) counts were collected and assessed to better understand the correlation of assay performance to the cycle threshold (Table 6).

TABLE 6
The performance of the VITROS ® SARS-CoV-2
Antigen test with positive results stratified by the
comparator method cycle threshold (Ct) counts

| VITROS ® | Comparator Method | |
|---|---|---|
| SARS-CoV-2 | (Positive by Ct category) | |
| Antigen Test | Positive (<30 Ct) | Positive (≥30 Ct) |
| Reactive | 48 | 1 |
| Non-reactive | 4 | 6* |
| PPA | 92.3% | 14.3% |
| Positive Agreement | 81.5-97.9% | 0.4-57.9% |
| (95% Cl) | | |

*One of the 6 mon-reactive results was also negative on an alternate RT-PCR method.

### Example 5. Clinical Concordance of VITROS® Nasal Vs RT-PCR Nasopharyngeal Results

The concordance of the disclosed VITROS® assay compared to the comparator RT-PCT nasopharyngeal results are presented in Tables 7A-B below.

TABLE 7A
VITROS ® SARS-CoV-2 Antigen Performance in RT-PCR
Positive and Negative Nasal Samples Collected Within
7 Days of Symptom Onset Against the Comparator Method

| ALL | RT-PCR Nasopharyngeal | | |
|---|---|---|---|
| VITROS ® Nasal | Detected | Not Detected | Total |
| Reactive | 49 | 0 | 49 |
| Non-reactive | 16 | 87 | 103 |
| Total | 65 | 87 | 152 |

PPA*: 75.4% (95% Cl: 63.1-85.2%)
NPA: 100.0% (95% Cl: 95.8-100.0%)
*The decreased PPA may be attributed to lower viral loads present in anterior nasal swabs when compared to NP swabs. Paired RT-PCR specimens demonstrated a PPA of 90.8% when comparing RT-PCR anterior nasal sample results to RT-PCR nasopharyngeal samples.

TABLE 7B
Positive results broken down
by days since symptom onset

| Days Since | Cumulative | Cumulative | |
|---|---|---|---|
| Symptom | VITROS ® | VITROS ® | |
| Onset | PCR Positive (+) | Reactive (+) | PPA |
| 0 | 2 | 1 | 50.0% |
| 1 | 8 | 7 | 87.5% |
| 2 | 19 | 17 | 94.4% |
| 3 | 34 | 29 | 93.5% |
| 4 | 39 | 32 | 91.4% |
| 5 | 54 | 42 | 85.7% |
| 6 | 62 | 47 | 82.5% |
| 7 | 65 | 49 | 83.1% |

The performance of the VITROS® SARS-CoV-2 Antigen test with positive results in Nasal Samples stratified by the comparator method C) counts in nasopharyngeal Samples were collected and assessed to better understand the correlation of assay performance to the cycle threshold in the different sample types (Table 8).

TABLE 8
The performance of the VITROS ® SARS-CoV-2
Antigen test with positive results stratified by the
comparator method cycle threshold (Ct) counts

| VITROS ® | Comparator Method | |
|---|---|---|
| SARS-CoV-2 | (Positive by Ct category) | |
| Antigen Test | Positive (<30 Ct) | Positive (≥30 Ct) |
| Reactive | 49 | 0 |
| Non-reactive | 9 | 7 |
| PPA | 84.5% | 0.0% |
| Positive | 72.6-92.7% | 0.0-34.6% |
| Agreement (95% Cl) | | |

### Example 6. Cross-Reactivity of Assay

The VITROS® SARS-CoV-2 antigen test was evaluated for potential microbial cross-reactivity using contrived samples in the absence and presence of SARS-CoV-2. Potentially cross-reactive organisms were spiked into solution at concentrations of greater than or equal to 10 CFU/ml for bacteria and greater than or equal to 10 pfu/ml for viruses. The results are summarized in Table 9 below.

TABLE 9

| | Non-Reactive | Spiked Reactive | Cross-Reactivity |
|---|---|---|---|
| Sample Category | Sample | Sample | (Y/N) |
| Human coronavirus 229E | Non-Reactive | Reactive | N |
| Human coronavirus OC43 | Non-Reactive | Reactive | N |
| Human coronavirus NL63 | Non-Reactive | Reactive | N |
| Influenza A H3N2 | Non-Reactive | Reactive | N |
| Influenza B | Non-Reactive | Reactive | N |
| Adenovirus (e.g., C1 Ad. 71) | Non-Reactive | Reactive | N |
| Human Metapneumovirus (hMPV) | Non-Reactive | Reactive | N |
| Parainfluenza virus 1-4 | Non-Reactive | Reactive | N |
| Enterovirus | Non-Reactive | Reactive | N |
| Respiratory syncytial virus | Non-Reactive | Reactive | N |
| Rhinovirus | Non-Reactive | Reactive | N |
| Hemophilus influenzae | Non-Reactive | Reactive | N |
| Streptococcus pneumoniae | Non-Reactive | Reactive | N |
| Streptococcus pyogenes | Non-Reactive | Reactive | N |
| Candida albicans | Non-Reactive | Reactive | N |
| Bordetella pertussis | Non-Reactive | Reactive | N |
| Mycoplasma pneumoniae | Non-Reactive | Reactive | N |
| Legionella pneumophila | Non-Reactive | Reactive | N |
| MERS-coronavirus | Non-Reactive | Reactive | N |
| Chlamydophila pneumoniae | Non-Reactive | Reactive | N |
| Staphylococcus epidermidis | Non-Reactive | Reactive | N |
| Staphylococcus aureus | Non-Reactive | Reactive | N |
| Pooled human nasal wash | Non-Reactive | Reactive | N |

To estimate the likelihood of cross-reactivity with SARS-CoV-2 virus in the presence of organisms that were not available for wet testing, in silico analysis using the Basic Local Alignment Search Tool (BLAST) managed by the National Center for Biotechnology Information (NCBI) was used to assess the degree of protein sequence homology.

No protein sequence homology was found between M. tuberculosis, P. jirovecii, or HCov-HKU1, thus cross-reactivity can be ruled out.

The comparison between SARS-CoV-2 nucleocapsid protein and SARS-CoV-1 shows homology of 90.52% and suggests that there will be significant cross reactivity in this test.

### Example 7. Substances that do not Interfere with Assay

The VITROS® SARS-CoV-2 Antigen test was evaluated for interference. Of the compounds tested, none was found to interfere with the clinical interpretation of the test in Non-reactive and weakly Reactive samples at the concentrations indicated in Table 10.

TABLE 10

| Interfering Substance | Active Ingredient | Concentration | |
|---|---|---|---|
| Human blood | Blood | 4% | |
| Hemoglobin | Hemolysate | 1000 | mg/dL |
| Purified mucin protein | Mucin protein | 5.0 | mg/mL (5%) |
| OTC Nasal Spray 1 | Oxymetazoline | 15% | |
| OTC Nasal Spray 2 | Fluticasone | 5% | |
| OTC Nasal Spray 3 | Triamcinolone | 5% | |
| OTC Nasal Spray 4 | Phenylephrine | 15% | |
| | hydrochloride | | |
| OTC Nasal Spray 5 | Budesonide | 5% | |
| | (glucocorticoid) | | |
| OTC Nasal Spray 6 | Saline | 15% | |
| OTC Nasal Spray 7 | Cromolyn | 15% | |
| OTC Nasal Wash | Alkolol | 10% | |
| OTC Nasal gel | Sodium chloride | 5% | |
| | (NeilMed) | | |
| Sore Throat | Benzocaine, Menthol, | 0.7 | g/mL (70%) |
| Nasal Spray | Phenol | | |
| Throat Lozenge | Menthol | 0.8 | g/mL (80%) |
| Anti-viral Drug 1 | Oseltamivir | 5.0 | μg/mL |
| Anti-viral Drug 2 | Zanamivir | 282.0 | ng/mL |
| Anti-bacterial, systemic | Tobramycin | 1.25 | ng/mL |
| Hemepathic Cold | Galphimia glauca, Luffa | 5% | |
| Remedy | operculata, Sabadilla | | |
| Antibacterial | Mupirocin | 10 | mg/mL |

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above described elements in all possible variations thereof is encompassed by the invention unless otherwise indicated herein or otherwise clearly contradicted by context.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

What is claimed is:

1. A method for the high thoughput testing of samples for the presence of SARS-CoV-2 nucleocapsid comprising:
providing a immunoassay system capable of performing high throughput assays;
reacting a patient sample with a capture antibody that recognizes the SARS-CoV-2 nucleocapsid, a detection antibody that recognizes the SARS-CoV-2 nucleocapsid at a location different from the capture antibody, and a means for detecting a complex comprising the capture antibody, the detection antibody, and the SARS-CoV-2 nucleocapsid; and
detecting the complex comprising the capture antibody, the detection antibody, and the SARS-CoV-2 nucleocapsid;
wherein the reacting and detecting steps are performed in the immunoassay system.

2. The method according the claim 1, wherein the immunoassay system capable of performing high throughput assays is an Ortho Clinical Diagnostics VITROS® device.

3. The method according to claim 1, wherein the capture antibody is associated with a solid support.

4. The method according to claim 1, wherein the capture antibody is a monoclonal antibody specific for the SARS-CoV-2 nucleocapsid.

5. The method according to claim 4, wherein the capture antibody is a rabbit monoclonal antibody.

6. The method according to claim 1, wherein the detection antibody is a monoclonal antibody specific for the SARS-CoV-2 nucleocapsid.

7. The method according to claim 5, wherein the detection antibody is a mouse monoclonal antibody.

8. The method according to claim 1, wherein the assay does not cross-react with coronaviruses other than SARS-CoV-1.

9. The method according to claim 1, wherein the detection antibody is in a conjugate, wherein each conjugate comprises about 100 horseradish peroxidase (HRP) molecules and 25 immunoglobulin molecules.

10. The method according to claim 1, wherein the patient sample is a nasopharyngeal swab or an anterior nasal swab.

11. A kit for the high thoughput testing of samples for the presence of SARS-CoV-2 nucleocapsid in a high throughput immunoassay system comprising:
a capture antibody that recognizes the SARS-CoV-2 nucleocapsid and a solid support associated therewith;
a detection antibody that recognizes the SARS-CoV-2 nucleocapsid at a location difference from the capture antibody;
a means for detecting a complex comprising the capture antibody, the detection antibody, and the SARS-CoV-2 nucleocapsid; and
instructions for performing the assay.

12. The kit according the claim 11, wherein the high throughput immunoassay system is an Ortho Clinical Diagnostics VITROS® device.

13. The kit according to claim 11, wherein the capture antibody is associated with a solid support.

14. The kit according to claim 11, wherein the capture antibody is a monoclonal antibody specific for the SARS-CoV-2 nucleocapsid.

15. The kit according to claim 14, wherein the capture antibody is a rabbit monoclonal antibody.

16. The kit according to claim 11, wherein the detection antibody is a monoclonal antibody specific for the SARS-CoV-2 nucleocapsid.

17. The kit according to claim 16, wherein the detection antibody is a mouse monoclonal antibody.

18. The kit according to claim 11, wherein the capture antibody and detection antibody do not cross-react with coronaviruses other than SARS-CoV-1.

19. The kit according to claim 11, wherein the detection antibody is in a conjugate, wherein each conjugate comprises about 100 HRP molecules and about 25 immunoglobulin molecules.
